(19) United States
(12) Patent Application Publication
(10) Pub. No.: US 2008/0297764 A1
(43) Pub. Date: Dec. 4, 2008

(54) Sensor for determining body parameters

(75) Inventors: Bernhard Scholler, Karlsruhe (DE); Mike Bernstein, San Ramon, CA (US); Thomas Magin, Schifferstadt (DE); Carola Schmidt, Henstedt Ulzburg (DE)

Correspondence Address: FRIEDRICH KUEFFNER, 317 MADISON AVENUE, SUITE 910, NEW YORK, NY, 10017, US

(73) Assignee: Weinmann Gerate fur Medizin GmbH + Co. KG

(21) Appl. No.: 11/985,351

(22) Filed: Nov. 13, 2007

(30) Foreign Application Priority Data
Nov. 13, 2006 (DE) 10 2006 053 688.6

(51) Int. Cl. G01N 33/48

(52) U.S. Cl. 356/41

(57) ABSTRACT

A sensor for measuring at least one body parameter, particularly blood and/or tissue parameters, is used for carrying out the measurements of electromagnetic radiation in the transmission or reflection methods, wherein the sensor uses at least one LED as a source of electromagnetic radiation. At least one photodetector is used as the receiving element. At least one LED is used in a non-invasive measurement of the parameters for ensuring a sufficiently high residual intensity of the radiation received by the photodetector and transmitted or reflected by the blood and/or tissue, wherein the LED has a light intensity of at least 200 millicandela and/or a light yield of at least 2 lumen/watt.

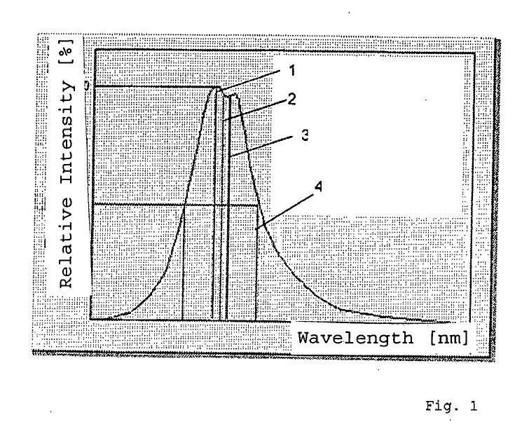

### BACKGROUND OF THE INVENTION

1. Field of the Invention

The present invention relates to a sensor for determining blood parameters, tissue parameters, or skin parameters, for example, of oxygen saturation SaO2, carbon monoxide saturation SaCO, hemoglobin concentration cHb, by means of electromagnetic waves using the transmission method or the reflection method. The method uses a carrier body which carries at least one transmission element and at least one receiving element and is composed at least over portions thereof of an elastic material, wherein the elastic material is arranged in such a way that in one state of operation it rests at least over portions thereof against a human body part and/or organ, preferably a finger, toe, tongue or earlobe.

2. Description of the Related Art

The determination of the oxygen saturation SaO2, the carbon monoxide saturation SaCO, or the hemoglobin concentration cHb of a patient frequently is of a high clinical relevance because a deviation from a desired value permits conclusions with respect to a critical state of the patient.

In pulse oximetry, the measurement of the pulse oximetrically determined oxygen saturation as SpO² is carried out by means of electromagnetic waves of different wavelengths which are radiated into the tissue of a patient. In this measurement, light diodes are frequently used as transmitters which emit light waves in the red and in the infrared ranges. Photosensitive receiving diodes measure the intensity of the light penetrating through the tissue and the blood vessels of the patient (transmission method) or the intensity of the reflected light (reflection method). Using the measured weakening of the reflections, the oxygen saturation in the blood can be computed.

The principle of the pulse spectroscopy uses, similar to the pulse oximetry, electromagnetic waves of different wavelengths. However, in pulse spectroscopy, always more than two wavelengths are used for determining additional parameters, such as oxygen saturation SaO2, carbon monoxide saturation SaCO, met hemoglobin saturation SaMet, sulf hemoglobin saturation SaSulf, hemoglobin concentration cHb. Method and apparatus for computing the parameters by means of pulse spectroscopy are described, for example, in German patent applications DE 103 21 338 A1, DE 102 13 692 A1, DE 10 2006 052 125 A1, DE 10 2006 053 975 A1.

Problems in the realization of apparatus which determine parameters by means of pulse spectroscopy occur particularly because, after radiating the electromagnetic waves into a body part, a large portion of the energy is absorbed in the tissue. This is particularly true for wavelengths above 1000 nm. Weakening in the tissue is particularly high in that range. The portion of reflected or transmitted light is accordingly very small. Previous experiments in realizing an apparatus failed because the signals available for evaluation are too small.

### SUMMARY OF THE INVENTION

Therefore, it is the primary object of the present invention to provide an apparatus in which the emitter and the detectors are selected and adjusted to each other in such a way that sufficiently high residual signal intensities are present for evaluation.

In accordance with the present invention, a sensor is used for measuring blood and/or tissue parameters by means of electromagnetic radiation in the transmission or reflection method, wherein at least one LED is used as the source of the electromagnetic radiation and at least one photodetector is used as the receiving element, wherein, in the non-invasive measurement of a blood and/or tissue parameter, wherein, in the non-invasive measurement of a blood and/or tissue parameter, at least one LED having a light intensity of at least 2000 millicandela (mCd) and/or a light yield of at least 2 lumen/watt is used for ensuring a sufficiently high residual intensity of the radiation received by the photodetector and the radiation transmitted or reflected by the blood and/or tissue.

The electromagnetic radiation is selected from one or more ranges of 150 nm±15%, 400 nm±15%, 460 nm±15%, 480±15%, 520±15%, 550 nm±15%, 560 nm±15%, 570 nm±15%, 580 nm±15%, 590 nm±15%, 600 nm±15%, 606 nm±15%, 617 nm±15%, 620 nm±15%, 630 nm±15%, 650 nm±15%, 660 nm±15%, 705 nm±15%, 710 nm±15%, 720 nm±15%, 775 nm±15%, 805 nm±15%, 810 nm±15%, 880 nm±15%, 905 nm±15%, 910 nm±15%, 950 nm±15%, 980 nm±15%, 1050 nm±15%, 1100 nm±15%, 1200 nm±15%, 1310 nm±15%, 1380 nm±15%, 1450 nm±15%, 1600 nm±15%, 1650 nm±15%, 1800 nm±15%, 2100 nm±15%, 2800 nm±15%.

The interval limits of ±15% is one embodiment. Interval limits in the range of ±10% are preferred and especially preferred is the interval limit in the range of ±5%, and especially preferred is an interval limit in the range of ±1%.

In addition to the LED, the source of electromagnetic radiation, i.e., the emitter can be presented as an LED as well as white light sources.

In accordance with the invention, only those LEDs are used which meet certain output characteristics. The LEDs must have a light intensity of at least 200 millicandela (mCd), preferably of 500 mCd, and particularly preferred at least 700 mCd. The light yield must be at least 3 lumen/watt, preferably at least 6 lumen/watt.

The selection of the LEDs and the detector material used depends on the parameter to be determined.

Preferred detectors are photodetectors of Si, Ge, InGaAs, AlGaAs GaAs, exemplified in the following detectors:

250-1100 nm UV reinforced Si photodiode, 1 cm² square active surface;

800-1700 nm Ge photodiode having 3 mm diameter as active surface;

800-1700 nm indium, gallium, arsenide InGaAs photodiode having 2 mm as diameter of active surface;

1-3 μm lead sulfide PbS photo conductor detector;

1-5 μm lead selenid PbSe photo conductor detector;

250-3000 nm dual detector with Si photodiode and lead sulfide PbS/Si photo conductor detector in sandwich construction;

1.5-5.5 μm indium antimonide InSb detector;

4-12 μm mercury-cadmium-telluride detector.

In accordance with a preferred embodiment, the detector is of sandwich construction. This makes possible a space-saving configuration especially for the two wavelengths measurement or for the multiple wavelengths measurement. The detector is composed of at least two detectors which are arranged one behind the other in a sandwich configuration. The detector is located in a closed housing. The detector which is first subjected to light absorbs a portion of the impinging light, wherein the remaining light penetrates this detector and is detected by the second detector located behind the first detector. The relationship between the two signals is a function of the wavelength. By providing the detectors, it is ensured that no mistake occurs as a result of the measurement of radiation which impinges from different directions on the measurement system.

The detector which the light impinges upon first absorbs the wavelengths portion of the impinging light having the longer wavelength portion, and the remaining shorter wavelength light penetrates the detector and is detected by the second detector located behind the first detector. For example, a Ge detector is used on an Si detector, wherein the detector detects wavelengths essentially of more than 1000 nm and the Si detector detects wavelengths of essentially below 1000 nm. Alternatively, different detector materials can be arranged next to each other. It is also being considered to split up the light radiation to detectors located next to each other by using a prism.

The detectors, particularly the detectors of sandwich construction, ensure a low black flow, low noise, and a high saturation flow. Consequently, a large dynamic range is made possible which is necessary in order to make it possible to carry out exact measurements of a large measurement in temperature range.

The sensors according to the invention have a storage element for storing codified data, wherein the codification of the type SHA type, which is a secure codification technology of the type Hash.

Preferred materials for manufacturing the sensor are silicon, rubber, or polyurethane, wherein the sensor can be manufactured by any suitable method, particularly injection molded or cast. A comparable elastic material is also suitable for this purpose. The transmitter and receiving elements can be glued onto the carrier body; however, particularly advantageous is a method in which the transmitting and receiving elements are surrounded by injection molded or cast material when manufacturing the carrier body. This simplifies cleaning and sterilization of the sensor.

The contact surface for a body part, for example, a finger, is dark in the sensors according to the present invention, preferably black or dark grey, so that scattered light by the contact surface is essentially avoided.

The contact surface for a body part, for example, a finger, is constructed essentially as a plane surface, wherein the optical elements, namely, emitter and detector, are at least over portions thereof raised above the level of the contact surface. This slightly raised arrangement results in a good contact between the body part and the emitter and/or a good contact between the body part and the detector.

In the sensor according to the present invention, the sensors are arranged in such a way that an essentially rectangular assembly surface is obtained. Preferably, the emitters are arranged in rows of 2×2 or 2×3 or 3×3, or 3×3.

It is also conceivable according to the present invention that the emitters have an essentially circular or rounded assembly surface.

The various features of novelty which characterize the invention are pointed out with particularity in the claims annexed to and froming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

The single FIGURE of the drawing schematically illustrates the wavelength-dependent relative light intensity of an LED.

### DETAILED DESCRIPTION OF THE INVENTION

While LEDs are monochromatic (asides from white LEDs), they still do not cast their light over a relatively wide spectrum. The drawing shows the relative light intensity of an LED at various wavelengths. The drawing also shows wavelengths which characterize a LED:

The peak wavelength (1) designates the wavelength of the intensity maximum.

The half value width characterizes the differences of the half value wavelengths (4) which the radiation intensity has dropped to 50% of the intensity maximum.

The center wavelength (2) characterizes the middle value of the two wavelengths for the half value width. As a rule, the center point wavelength (2) is because of the asymmetrical curve shape not identical to the gravity center point wavelength (3).

The gravity center wavelength (3) takes into consideration the entire spectral intensity distribution. The gravity center wavelength divides the curve into two areas having the same integral intensity. The outputs to the left and right of the center gravity wavelengths are equal. The gravity center point wavelength is usually not identical to the center point wavelength because of an asymmetrical curve shape.

The light yield is a measurement for the effective conversion of electrical energy into light energy. The efficiency of the LED according to the invention is about 2 to 50 lm/W.

For determining the carbon monoxide saturation SaCO in the blood, an LED is used having a gravity center wavelength of 606 nm±15% and/or with a light intensity of at least 200 millicandela (mCd), preferably of at least 500 mCd, and especially preferred of at least 700 mCd and/or a light yield of at least 6 lumen/watt, preferably of at least 9 lumen/watt.

The emitters (LEDs) according to the invention emit in a wavelength range of 606 nm±15%, and/or 660 nm±15%, and/or 905 nm±15%. In one embodiment, at least two LEDs are used for emitting the radiation from a wavelength range of 606 nm±15% and/or 660 nm±15% and/or 905 nm±15%. Preferred are the LEDs when connected in series or parallel.

Used as the detector material are Si and/or Ge and/or AlGaAS and/or GaAs detectors.

In accordance with another embodiment, a sensor is used for determining SaCO which detects by means of two LED the radiation of the wavelength ranges of 606 nm±15%, and/or 660 nm±15%, and/or 905 nm±15% and a detector which detects at least two of the wavelengths ranges of 606 nm±15%, and/or 660 nm±15%, and/or 905 nm±15%, particularly by having different detector materials arranged in the area of the sensor.

For determining the parameters cHb, at least one LED having a wavelength of preferably greater than 1000 nm is used, wherein the LED has a light intensity of at least 200 millicandela (mCd), preferably at least 500 mCd, particularly preferred of at least 700 mCd and whose light yield is at least 3 lumen/watt.

For determining the hemoglobin concentration cHb in the blood, alternatively an LED having a gravity center wavelength of greater than 1000 nm, particularly of 1450 nm±15%, and with a light intensity of at least 100 millicandela (mCd), preferably of at least 200 mCd, especially preferred at least 700 mCd and/or a light yield of at least 6 lumen/watt, preferably at least 9 lumen/watt, is used.

In accordance with another embodiment, a sensor is used for determining cHb which emits by means of at least two LEDs radiation of the wavelength ranges of 660 nm±15% and/or 1450 nm±15% and/or 905 nm±15% and/or 805 nm±15%, and a detector which detects at least two of the wavelength ranges of 660 nm±15% and/or 1450 nm±15% and/or 905 nm±15% and/or 805 nm±15%, in particular by having different detector materials arranged, for example, in sandwich construction, in the area of the sensor. Preferred is a detector which contains at least one germanium for the determination of cHb. Alternatively, Si and/or AlGaAS and/or GaAs and/or InGas and/or PbS detectors and/or GeTe are provided.

For determining cHb and SaCO, a preferred embodiment of the invention provides that a combination sensor is used which emits radiation by means of at least three LEDs having a wavelength range of 606 nm±15% and/or 660 nm±15% and/or 1450 nm±15% and/or 905 nm±15% and/or 805 nm±15%, and a detector which detects at least two of the wavelength ranges of 606 nm±15% and/or 660 nm±15% and/or 1450 nm±15% and/or 905 nm±15% and/or 805 nm±15%, particularly by arranging different detector materials in the area of the sensor. In this regard, detectors are preferred which are constructed according to the sandwich principle.

The invention can be used, for example, in a portable patient monitoring system which is battery operated but can also be connected to a mains connection. The weight is preferably below 200g, and the volume is preferably below 600 ccm. The monitor according to the present invention is distinguished by the integration of at least two of the following parameters: EKG, SpO2, SaCO, cHb, and/or bilirubin.

An application is also conceivable, for example, in gynecology as an additional parameter in a CTG, or as a supplemental parameter cHb in connection with dialysis or as a supplemental parameter in breathing ventilation, or as a supplemental parameter for checking an infusion, for example, with an infusion pump.

While specific embodiments of the invention have been shown and described in detail to illustrate the inventive principles, it will be understood that the invention may be embodied otherwise without departing from such principle

We claim:

1. A sensor for measuring blood and/or tissue parameters using electromagnetic radiation by means of transmission or reflection methods, the sensor comprising at least one LED as a source of electromagnetic radiation, and a photo detector as a receiving element, further comprising, in a non-invasive measurement of a blood and/or tissue parameter and for ensuring a sufficiently high residual intensity of the radiation received by the photo detector and transmitted or reflected by the blood and/or tissue, wherein the at least one LED has a light intensity of at least 200 millicandela and/or a light yield of at least 2 lumen/watt.

2. The sensor according to claim 1, wherein the LED emits at least one of the wavelengths selected from the group 150 nm±15%, 400 nm±15%, 460 nm±15%, 480 nm±15%, 520 nm±15%, 550 nm±15%, 560 nm±15%, 570 nm±15%, 580 nm±15%, 590 nm±15%, 600 nm±15%, 606 nm±15%, 617 nm±15%, 620 nm±15%, 630 nm±15%, 650 nm±15%, 660 nm±15%, 705 nm±15%, 710 nm±15%, 720 nm±15%, 775 nm±15%, 805 nm±15%, 810 nm±15%, 880 nm±15%, 905 nm±15%, 910 nm±15%, 950 nm±15%, 980 nm±15%, 1050 nm±15%, 1100 nm±15%, 1200 nm±15%, 1310 nm±15%, 1380 nm±15%, 1450 nm±15%, 1600 nm±15%, 1650 nm±15%, 1800 nm±15%, 2100 nm±15%, 2800 nm±15%.

3. The sensor according to claim 1, further comprising, for determining the carbon monoxide saturation SaCO in the blood, at least one LED having a gravity center wavelength in the range of 606 nm±15% or 660 nm±15% or 805 nm±15%, and with a light intensity of at least 200 millicandela and a light yield of at least 2 lumen/watt.

4. The sensor according to claim 1, comprising, for determining the hemoglobin concentrations in the blood, at least one LED having a gravity center wavelength in the range of 1450 nm±15% and/or 905 nm±15% and/or 805 nm±15%, and a light intensity of at least 100 millicandela and a light yield of at least 2 lumen/watt.

5. The sensor according to claim 1, further comprising, for determining cHb, by means of a wavelength of 1450 nm±15%, an LED having a light intensity of at least 200 millicandela, and whose light yield is at least 6 lumen/watt.

6. The sensor according to claim 5, wherein the light intensity of the LED is at least 500 mCd.

7. The sensor according to claim 5, wherein the light intensity of the LED is at least 700 mCd.

8. The sensor according to claim 1, comprising a detector of a material selected from the group consisting of Si, Ge, InGaAs, AlGaAs, PbS, PbSe, InSb.

9. The sensor according to claim 1, comprising a detector of sandwich construction selected from at least two of the materials Si, Ge, InGaAs, AlGaAs, PbS, PbSe, InSb.

10. The sensor according to claim 1, comprising a detector of sandwich construction, wherein the detector material of the layer to which light is emitted first has a wavelength of essentially greater than 1000 nm, and a detector material located behind detects essentially wavelengths smaller than 1000 nm.

11. The sensor according to claim 1, comprising a photodetector of Ge and/or InGaAs and/or AlGaAs for detecting wavelengths in the range of greater than 1000 nm.

12. The sensor according to claim 1, comprising at least one photodetector of the material Si and/or Ge for detecting wavelengths in the range of greater than 100 nm.

13. The sensor according to claim 1, wherein the sensor is comprised of an upper part and a lower part, and wherein the upper part and the lower part are adapted to receive at least in one state of operation a human body part, and wherein at least one cushion is provided in an area between upper part and/or lower part, wherein the cushion is arranged adjacent to the human body part, and wherein the cushion is black or of a dark color.

14. The sensor according to claim 1, wherein at least three sources of electromagnetic radiation are arranged essentially as corner points of a spatial arrangement in the area of the sensor in such a way that the at least three sources of electromagnetic radiation are in at least one state of operation less than one centimeter away from the human body part.

15. The sensor according to claim 14, wherein at least three sources of electromagnetic radiation are arranged essentially as corner points of a spatial arrangement and wherein at least one additional source of electromagnetic radiation is arranged essentially in the middle between the other sources.

16. The sensor according to claim 1, wherein at least four sources of electromagnetic radiation are arranged essentially as corner points of a spatial arrangement and wherein an additional source of electromagnetic radiation is arranged essentially in the middle between the other sources.

17. The sensor according to claim 1, wherein, in at least one state of operation, a safe Hash algorithm is used for recognizing the sensor.

18. A method of selecting a suitable LEDs in a planned use of the LEDs for determining blood and/or tissue parameters, the method comprising determining by means of a spectrometer the criteria half value width, and/or center wavelength and/or peak wavelength of the LEDs, wherein defined limit values are present for the half value width and/or the center wavelength and/or the peak wavelengths, and using the LED when the LED is at least with respect to one criteria in the range of the accepted limit values.

19. The method according to claim 18, comprising using the method as a control method of an automated sorting plant.
